(19) United States
(12) Patent Application Publication
(10) Pub. No.: US 2024/0408311 A1
(43) Pub. Date: Dec. 12, 2024

(54) METHODS AND DEVICE FOR FILLING ONE OR MORE SYRINGES

(71) Applicant: SJJ Solutions B.V., Delft (NL)

(72) Inventors: Sander SCHOOTSTRA, Rijswijk (NL); Joost SCHOOTSTRA, Delft (NL); Jasper SCHOOTSTRA, Oegstgeest (NL)

(21) Appl. No.: 18/699,399

(22) PCT Filed: Oct. 6, 2022
(86) PCT No.: PCT/NL2022/050562

§ 371 Date: Apr. 8, 2024

(30) Foreign Application Priority Data
Oct. 6, 2021 (NL) 2029329

(51) Int. Cl. A61M 5/31; A61J 1/06; A61J 1/14; A61J 1/20

(52) U.S. Cl. CPC A61M 5/31; A61J 1/1468; A61J 1/2048; A61J 1/2075; A61J 1/2079; A61J 1/2096; A61J 1/065; A61M 2005/3114; A61M 2005/3123

(57) ABSTRACT

Methods and devices are provided for filling one or more syringes with a liquid, essentially without air being trapped with the liquid in the one or more syringes. A temporary storage volume—a buffer member—is used to temporarily hold the liquid and allow air to be removed from the liquid in the syringe. The size and/or shape of the temporary storage volume—also referred to as intermediate volume—allows the air to be removed more easily compared to the size and/or shape of a main volume of the one or more syringes.

### TECHNICAL FIELD

The aspects and embodiments thereof relate to the field of filter needles and syringes for administering liquid medication.

### BACKGROUND

Liquid medication may be supplied in glass ampoules. These ampoules are usually broken open by hand and the medication is then drawn up into a syringe. Particularly when the medication is administered intravenously, patient safety issues may occur if the liquid medication is contaminated with glass micro-particulate or miniscule glass shards originating from the breaking of the ampoule.

Liquid medication may be supplied in vials which are sealed with a rubber stopper. Coring is a known problem with these vials, wherein a small piece of the rubber stop breaks after puncturing the rubber stopper with a needle. This small piece of rubber may contaminate the liquid medication. As the piece of rubber may be very small, it may remain unnoticed and may cause harm to a patient when the piece of rubber is injected together with the liquid medication.

To filter out any contaminants, such as glass micro-particulate or rubber pieces, a filter needle may be used for drawing up the medication from the glass ampoule or vial into the syringe. The filter needle for example comprises a 5 micron filter, which catches the glass or rubber particles as the liquid medication is pulled through it.

After the liquid medication has been drawn into the syringe, the filter needle is discarded and a non-filter needle is coupled to the syringe in order to administer the medication.

### SUMMARY

It has been observed that when using a needle—in particular but not limited to a filter needle—to pull liquid medication into a syringe, air is also drawn into the syringe. This air needs to be removed from the syringe prior to administering the liquid medication. Pointing the syringe upwards and tapping the syringe may remove air trapped in the liquid medication in the syringe.

Instead of liquid medication, any other liquid may also be drawn into a syringe. For other liquids, it may also be required to remove air trapped in the liquid inside the syringe. This especially holds for expensive or rare liquids, with a high cost per unit, in particular per unit volume like litre or millilitre, which makes wasting the liquid even less desirable. Examples of expensive and/or rare liquids are liquid medicaments, in particular vaccines or medicaments for intravitreal injection, or any other liquid substance.

It has been observed that when the diameter of the syringe, in particular of the main volume of the syringe, is too small, air may remain trapped with the liquid medication inside the syringe. This may for example be caused by the viscosity of the liquid medication. It is preferred to provide for a method and/or device to administer filtered liquid medication essentially without air trapped therein, in particular using a syringe with a small diameter, in particular with a small inner diameter.

A first aspect provides a method of filling a syringe with a liquid. The method comprises the steps of filling an intermediate volume of a buffer member with the liquid, optionally emptying a main volume of a syringe, drawing the liquid from the intermediate volume of the buffer member into the main volume of the syringe such that the main volume is essentially free of air, and disconnecting the buffer member from the syringe.

The liquid may in general be supplied in any liquid container such as a vial, an ampoule, a syringe, or a bottle. The liquid container may comprise or consist of glass, plastic (organic polymer), metal, any other material, or any combination thereof. A liquid container may for example comprise approximately 0.1 mL-4 mL, but alternatively also 15 mL of liquid, or larger volumes such as 30 mL or more, or even 100 mL or more.

The intermediate volume of the buffer member may be filled by drawing the liquid into the intermediate volume using a syringe connected to the buffer member. The syringe may be operated by a person, or by a machine.

As an option, the liquid is drawn into the intermediate volume through a filter needle. By passing through a filter of the filter needle, the liquid becomes a filtered liquid. Being a filtered liquid does not necessarily mean that particulate has been filtered from the liquid, for example in cases where there was no particulate in the unfiltered liquid which had to be filtered out. Filtering may be a precautionary measure.

In the present disclosure, the term fluid may mean a liquid, a gas, or a combination thereof. The liquid may in particular be a liquid medicament, supplied in and drawn from a glass ampoule. The liquid medicament may be intended for intravenous injection into a human or animal. The gas may in particular be air.

The method may further comprise a step of connecting the buffer member to the syringe, in particular in an essentially liquid-tight manner such that liquids may be transferred between the buffer member and the syringe without leakage in normal use. In other embodiments, the buffer member may already be connected to the syringe.

A non-filter needle may be connected to the syringe after disconnecting the buffer member from the syringe. Via the non-filter needle, for example, the liquid inside the syringe may be injected, for example into a human or animal, in particular into an eye of a human or animal.

The method may as an option comprise a step of connecting the filter needle to the buffer member. When a filter needle is connected to a buffer member, also a syringe may be connected to the same buffer member. As such, the syringe may be used for drawing a liquid into the buffer member via the filter needle.

The liquid medicament may be filled into the intermediate volume from a glass ampoule or from a vial with a rubber stopper, wherein for example a syringe is lowered into the ampoule or vial below the liquid level in order to draw liquid up into the intermediate volume.

Liquid may first be drawn into the main volume of the syringe, subsequently pushed into the intermediate volume of the buffer member, and then drawn from the intermediate volume of the buffer member back into the main volume of the syringe.

The volume of filtered liquid drawn into the main volume of the syringe may be less than 60 mL, less than 10 mL, less than 0.5 mL, less than 0.2 mL or even less than 0.1 mL or less than 0.05 mL. Especially when using syringes with a small main volume, trapped air may be hard to remove from the syringe, for example by tapping the syringe with the tip pointed upward.

A second aspect provides a method of filling a plurality of syringes with a liquid, which as with the method according to the first aspect uses a buffer member with an intermediate volume to reduce or even fully prevent air becoming trapped in the main volumes of the plurality of syringes.

The method according to the second aspect comprises the steps of filling an intermediate volume of a buffer member with the liquid, in particular a liquid medicament drawn from a glass ampoule, vial, or other liquid container, for example by pulling back a plunger of a primary syringe of which a main volume is in liquid communication with the intermediate volume of the buffer member. The method further comprises connecting one or more secondary syringes to the buffer member, in particular one-by-one, and drawing liquid from the intermediate volume into main volumes of the one or more secondary syringes.

The primary syringe may also be filled by drawing liquid from the intermediate volume into its main volume. One, more, or all of the primary syringe and the secondary syringes may be similarly shaped and sized.

The method may further comprise a step of connecting a filter needle to the buffer member, and wherein a liquid is drawn through the filter needle into the intermediate volume. By passing through the filter needle, the liquid becomes filtered liquid.

As an example, between 0.01 to 0.5 ml, in particular between 0.05 to 0.2 ml, of liquid may be drawn into the main volumes of the secondary syringes. As a further example, between 0.02 to 5.0 ml, in particular between 0.1 to 1.0 ml, and more in particular 0.1 or 4.0 mL of liquid may be drawn into the intermediate volume of the buffer member.

A third aspect provides a kit of parts for forming a syringe assembly, with which at least parts of a method according to the first or second aspect may be performed.

The kit of parts comprises a syringe with a main volume for receiving a liquid, a buffer member, comprising a buffer body defining an intermediate volume for receiving a liquid into, a fluid inlet into the intermediate volume, and a vent opening for venting air from the intermediate volume, wherein the buffer member is connectable to the syringe to form the syringe assembly, and, in a connected state, the main volume and the intermediate volume are in fluid communication. By virtue of being in fluid communication, fluids such as liquid medicament and/or air may be transported between the main volume and the intermediate volume.

The syringe may comprise an elongated syringe body defining the main volume. As an option, a maximum cross-sectional area of the intermediate volume of the buffer body is larger than a maximum cross-sectional area of the main volume of the syringe in a direction perpendicular to an elongation direction of the syringe body, in particular at least two times larger. A maximum cross-sectional area may for example be defined by a maximum diameter in case of an approximately circular cross-sectional area.

Additionally or alternatively, the maximum cross-sectional area of the main volume may be less than 25 mm², for example less than 20 mm², as another example less than 17 mm² and as yet another example less than 15 mm² and the maximum inner cross-sectional area of the intermediate volume is less than 2·10³ mm², for example equal to or less than 1.2·10³ mm², equal to or less than 5.7·10² mm² or equal to or less than 1.8·10² mm², corresponding to an inner diameter of 39 millimetres, 27 millimetres and 15 millimetres.

At least part of an inner surface, or an entire inner surface arranged to contact liquid medicament in use, of the buffer body facing the intermediate volume may be a hydrophobic surface.

A hydrophobic surface may be defined as a surface which has a physical property of repelling water. A surface may be hydrophobic by virtue of the material providing the surface consisting of or comprising one or more hydrophobic materials, and/or by any structural or chemical modification of the surface to make the surface hydrophobic. A hydrophobic surface may in embodiments be provided by a hydrophobic coating on a substrate surface, for example part of the buffer member, which substrate surface is not necessarily hydrophobic.

To prevent contamination of liquid inside the intermediate volume, in particular liquid medicament, an inner surface of the buffer body facing the intermediate volume may be essentially free of liquid lubricants such as silicone oil.

A size of the intermediate volume may approximately correspond to more than two times a size of the main volume, preferably even five times or more. As such, in a single intermediate volume, sufficient liquid may be stored for filling a plurality of main volumes of a plurality of syringes. A size may be understood as a storage volume available for storing fluid, in particular liquid.

When, in use, the fluid inlet and the vent opening are aligned in an approximately horizontal plane, the buffer member may be arranged for holding a sub-volume of fluid, in particular liquid, in the intermediate volume below the fluid inlet and the vent opening, which sub-volume may approximately correspond to at least 0.2 times, 0.5 times, one time, or at least two times the size of the main volume of the syringe, and preferably even five times or more. The sub-volume may be arranged to contain a liquid in the buffer member when disconnecting a first syringe after filling the first syringe, and connecting a second syringe to be filled with liquid from the buffer member.

A fourth aspect provides the buffer member, providing the intermediate volume for temporarily storing the liquid, in particular the liquid medicament, in. The buffer member may be comprised by a kit of parts according to the third aspect, and may be used in performing at least part of a method according to the first and/or second aspect.

### BRIEF DESCRIPTION OF THE FIGURES

In the figures,

FIG. 1A shows a syringe;

FIG. 1B depicts the syringe of FIG. 1A after liquid medicament is drawn into the main volume of the syringe;

FIG. 2A shows an embodiment of a kit of parts in an assembled state;

FIG. 2B depicts the kit of parts of FIG. 2A after the plunger has been pressed down;

FIG. 3A depicts an embodiment of a kit of parts;

FIG. 3B shows the syringe of FIG. 3A with the buffer member disconnected;

FIGS. 4A and 4B schematically show in a longitudinal section view an embodiment of a buffer member, in two orientations;

FIGS. 4C and 4D show embodiments of a buffer member;

FIG. 5A shows a particular option wherein the filter needle can be connected to the buffer member; and

FIG. 5B shows an optional situation wherein a needle such as the filter needle is connected to the buffer member.

### DETAILED DESCRIPTION OF THE FIGURES

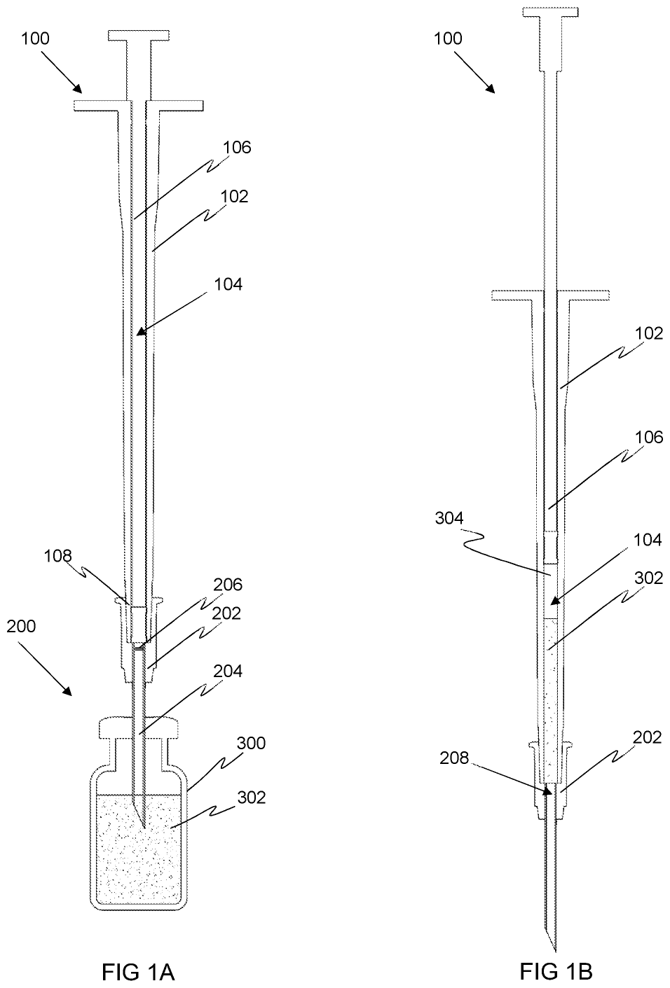

In general, FIGS. 1A-5B show schematic cross-sections of different assemblies which may be formed by a kit of parts and/or components which may be comprised by a kit of parts. In particular, the cross-sections are longitudinal sections along a typical elongation direction of the syringe. It will be understood that being schematic representations, different components may or may not be to scale, relative to each other. Some components may only have been provided with a reference numeral in one or some of the figures, while they are also present in other figures.

FIG. 1A shows a syringe 100, comprising an elongated syringe body 102. The syringe body 102 defines a main volume 104 therein. Inserted into the main volume 104 is a plunger 106, which can be moved up and down through the main volume 104 respectively for drawing in fluid such as liquid or air into the main volume 104 and for pushing fluid out of the main volume 104.

Liquid-tightly attached to the syringe 100, in particular to a tip 108 thereof, is a filter needle 200. The filter needle 200 comprises a filter needle body 202 with which the filter needle 200 is attached to the syringe 100, and a hollow needle 204 arranged to be inserted in to an ampoule. Positioned inside the filter needle body 202 is a filter 206 arranged to filter out particles from a flow of liquid drawn through the filter needle 200.

FIG. 1A depicts the syringe 100 prior to a liquid medicament 302 being drawn from a vial 300 as an example of a liquid container. FIG. 1B depicts syringe 100 after the liquid medicament 302 is drawn into the main volume 104 of the syringe 100. As schematically depicted in FIG. 1B, next to the liquid medicament 302, also air 304 has been drawn into the main volume 104. The air 304 may for example have originated from a dead volume 208 inside the filter needle body 202.

The air 304 may for example be trapped between the plunger 106 and the liquid medicament 302. Air 304 may additionally or alternatively be trapped within the liquid medicament 302, for example as one or more bubbles.

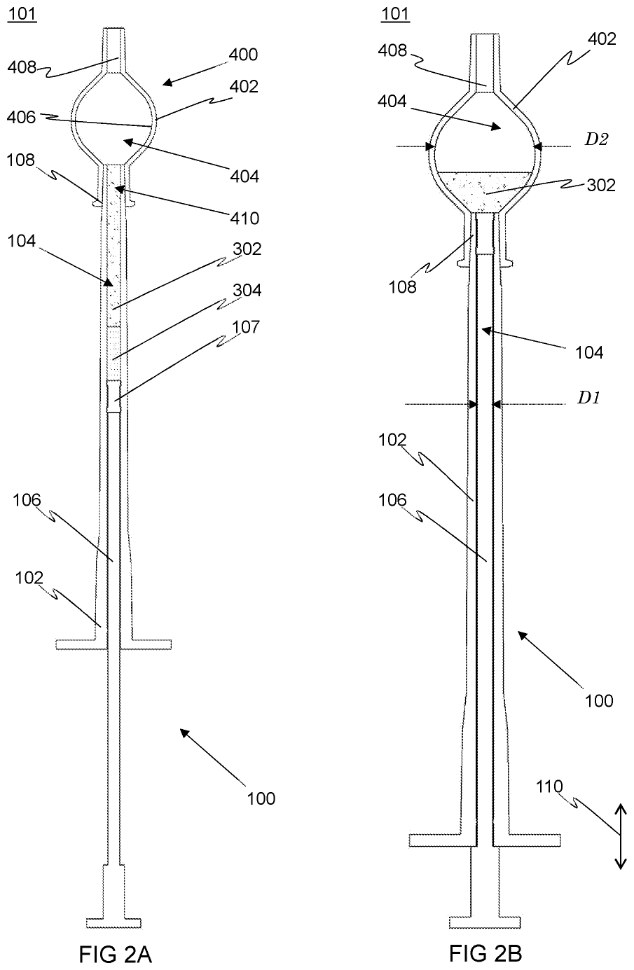

FIG. 2A shows an embodiment of a kit of parts 101 in an assembled state, forming a syringe assembly, comprising a syringe 100 and a buffer member 400. The buffer member 400 is liquid-tightly connected to the tip 108 of the syringe, for example via a slip fit connection such as a Luer slip connection. In the example of FIG. 2A, the buffer member 400 is hence slid over the tip 108 of the barrel of the syringe 100. However, other examples of how the buffer member 400 is liquid-tightly connected to the top 108 of the syringe are envisioned as well. For example, a Luer-lock or other threaded connection may be used, or part of the buffer member 400 may be pushed into the syringe 100. In different embodiments, the syringe 100 or the buffer member 400 may provide a male part for the connection, and the other may provide a female part for the connection.

The buffer member 400 comprises a buffer body 402 defining an intermediate volume 404 therein. The buffer body 402 has a vent opening 408 allowing passage of fluid, in particular air and/or liquid medicament, through the buffer body 402. The buffer body 402 also has a fluid opening 410 allowing passage of fluid, in particular air and/or liquid medicament, through the buffer body 402, in particular into and/or out of the intermediate volume 404.

At least part of an inner surface 406 of the buffer body 402 may be a hydrophobic surface. Additionally, or alternatively, at least part of the buffer body 402 may be tapered towards the fluid opening 410 of the buffer body 402.

FIG. 2B depicts the kit of parts 101 of FIG. 2A after the plunger 106 has been pressed down, i.e. moved generally towards the tip 108. As the plunger 106 is pressed down, the liquid medicament 302 and the air 304 inside the main volume 104 of the syringe is pushed into the intermediate volume 404 of the buffer member 400, via the fluid opening 410 of the buffer member 400.

By virtue of the shape of the intermediate volume 404, the air 304 which had been trapped in the syringe 100 may become released and may at least partially vent out of the intermediate volume 404 through the vent opening 408.

In FIG. 2B, a main volume diameter D1 is indicated, which defines a cross-sectional area of the main volume 104 in a plane perpendicular to an elongation direction 110 of the syringe 100. The elongation direction 110 generally corresponds to a direction of movement of the plunger 106 through the syringe body 102.

Also, in FIG. 2B, an intermediate volume diameter D2 is indicated, which in this example corresponds to a maximum diameter of the intermediate volume 404 regarded in a plane perpendicular to elongation direction 110, indicated in FIG. 2B with arrow 110.

It will be appreciated that diameters D1 and D2 are only an example, and that the cross-section of neither the main volume 104 nor the cross-section of the intermediate volume 404 is not necessarily a circular cross-section.

In general, the cross-section of the main volume 104, which may be defined by a passage through the syringe body 102, may be essentially constant. For example, the cross-section of the main volume 104 may have a circular shape, and a cross-section of an outer end 107 of the plunger 106 may correspond to the cross-section of the main volume 104.

In general, the cross-section of the intermediate volume 404 may be at least partially non-constant when regarded in a direction parallel to the elongation direction 110.

As schematically depicted in FIGS. 2A and 2B, the size of the intermediate volume 404, for example expressed in mL, may approximately correspond to the size of the main volume 104. In other embodiments, the size of the intermediate volume 404 may approximately correspond to more than the size of the main volume 104, in particular two times or more, three times or more, five times or more, ten times or more, or even fifteen times or more. Further examples of the size of the intermediate volume are between 0.01 mL and 16 mL, between 0.05 mL and between 3 mL and 23 mL. This may allow multiple syringes 100 to be filled from a single intermediate volume 404.

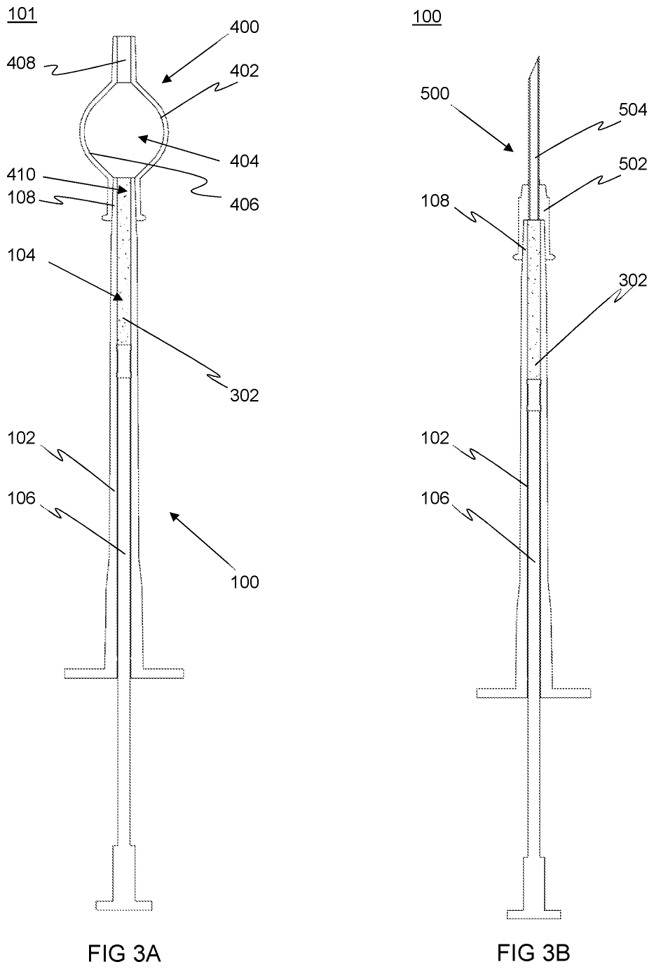

FIG. 3A depicts an embodiment of a kit of parts 101, after the liquid medicament 302 has been drawn back into the main volume 104 of the syringe 100, for example by having pulled back the plunger 106 in a direction away from the tip 108. The intermediate volume 404 may now be essentially empty, i.e. essentially only filled with air. Alternatively, liquid may be left in the intermediate volume 404 for filling one or more further syringes. The inner shape of the buffer body may be shaped to prevent liquid from becoming trapped in the intermediate volume 404, for example by virtue of being at least partially tapered towards the syringe 100 and/or by virtue of a hydrophobic property of at least part of the inner surface 406.

As schematically depicted in FIG. 3A, the main volume 104 of the syringe 100 is now essentially free of air. Because the liquid medicament 302 has as an option previously been passed through the filter needle 200, the liquid medicament 302 also less likely contains any glass, rubber, or other undesired particulate compared to liquid medicament 302 which has not passed through a filter needle.

FIG. 3B shows the syringe 100 of FIG. 3A with the buffer member 400 disconnected. The buffer member 400 was as an example connected to the syringe 100 with a threaded connection, such as a Luer lock connection. Instead, now an intravenous needle 500 as an example of a non-filter needle is connected to the tip 108 of the syringe 100. The needle 500 comprises a needle body 502 within which a sharp hollow needle end 504 is inserted.

During the method, an example of which is depicted in FIGS. 1A-3B, no, essentially no, or a minimum amount of liquid medicament is spilled. This may be particularly advantageous when the liquid medicament is costly and/or scarce.

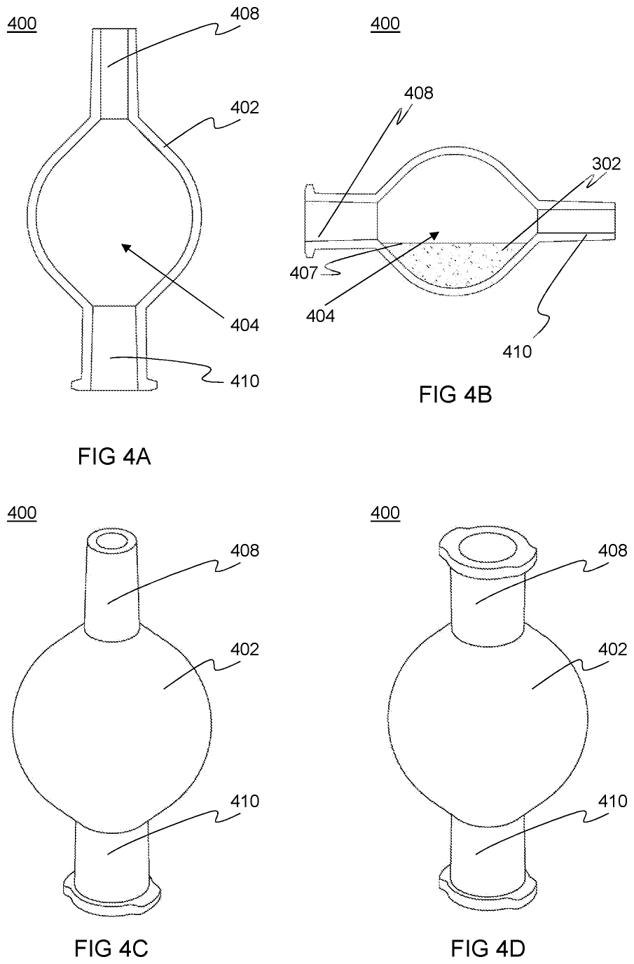

FIGS. 4A and 4B schematically show in a longitudinal section view an embodiment of a buffer member 400, in two orientations. FIG. 4A shows the buffer member 400 in an upright position, and FIG. 4B shows the buffer member rotated 90 degrees counter-clockwise with respect to FIG. 4A.

In FIG. 4B, the buffer member 400 is depicted with the intermediate volume 404 partly filled with a liquid, in particular a liquid medicament 302. The orientation of the fluid level of the liquid medicament 302 in this orientation generally corresponds to the horizon—i.e. perpendicular to gravity.

In general, a single buffer member 400 may be used for filling a plurality of syringes 100. One by one, multiple syringes such as secondary syringes may be connected to the buffer member 400 for drawing the liquid 302 out of the intermediate volume 404.

In-between connecting syringes 100 to the buffer member 400, the buffer member 400 may be held in the orientation shown in FIG. 4B to prevent liquid 302 from spilling out of the intermediate volume 404, for example through the fluid inlet 410 and/or the vent opening 408. As such, in the orientation shown in FIG. 4B, the fluid inlet 410 and the vent opening 408 are aligned in an approximately horizontal plane. An upper boundary 407 of a sub-volume is approximately indicated by a maximum liquid level for the orientation of FIG. 4B before liquid 302 would leak out of one or both of the fluid inlet 410 and the vent opening 408.

FIG. 4C shows the embodiment of the buffer member 400 of FIG. 4A in a perspective view. FIG. 4D shows another embodiment of a buffer member 400, indicating that embodiments are envisioned wherein one or both of the vent opening 408 and the fluid inlet 410 may be male fittings, and/or one or both of the vent opening 408 and the fluid inlet 410 may be female fittings, in any combination.

A buffer body 402 may be generally monolithic, or may comprise multiple parts which can be permanently or temporarily connected.

As can be seen in FIGS. 4A-4D, and what can be applied generally to any buffer member, the fluid inlet 410 may be formed by a protrusion protruding from the buffer body 402 and/or the vent opening 408 may be formed by a protrusion protruding from the buffer body 402. The two protrusions, when present, may extend in opposite directions away from each other.

A protrusion protruding from the buffer body 402 may be used for connecting a syringe 100 and/or a needle such as a filter needle or non-filter needle to, preferably in an essentially liquid-tight connection.

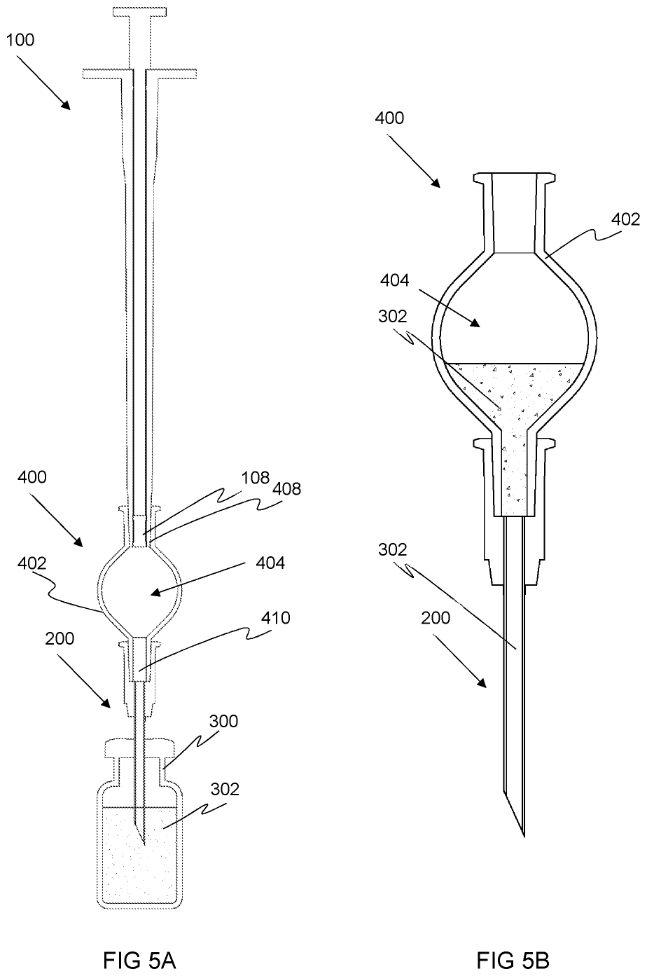

FIG. 5A shows a particular option, which may be readily combined with any embodiment of the kit of parts, wherein the filter needle 200 can be connected to the buffer member 400, for example to the fluid inlet 410, for example with a threaded connection. As such, prior to drawing up liquid medicament 302 from the vial 300 or any other liquid container such as an ampoule or another syringe, the buffer member 400 may be connected between the syringe 100, in particular the tip 108, and the filter needle 200. In such an example, the air vent 408 may also allow passage of the liquid medicament into the syringe 100.

FIG. 5B shows an optional situation wherein a needle such as the filter needle 200 or any other needle is connected to the buffer member 400, which contains a volume of liquid 302 in the intermediate volume 404. Liquid 302 may also be present in part of the filter needle 200. For example the filter or any other part of the filter needle 200 may prevent or reduce leakage of liquid 302, such that at least a majority of the liquid 302 remains contained in the intermediate volume 404, also when the filter needle 200 points downwards in a direction of gravity.

As such, multiple syringes 100 may be filled using liquid from the single buffer member 400 while the filter needle 200 remains attached to the buffer member 400, and the filter needle 200 prevents leakage out of the buffer member 400. Alternatively to using the filter needle 200, any other type of stop, valve or other seal or seals may be used to seal one or both of the fluid inlet 410 and the vent opening 408.

In the description above, it will be understood that when an element is referred to as being connect to another element, such as a syringe and a needle, or a syringe and a buffer member, the element is either directly connected to the other element, or intervening elements may also be present. Also, it will be understood that the values given in the description above, are given by way of example and that other values may be possible and/or may be strived for.

In general, one or two syringes may be connected simultaneously to a single buffer member for transferring fluid, such as a liquid, from one syringe to the other via the buffer member.

In summary, methods and devices are provided for filling one or more syringes with a liquid, essentially without air being trapped with the liquid in the one or more syringes. A temporary storage volume—a buffer member—is used to temporarily hold the liquid and allow air to be removed from the liquid. The size and/or shape of the temporary storage volume—also referred to as intermediate volume—allows the air to be removed more easily compare to the size and/or shape of a main volume of the one or more syringes.

It is to be noted that the figures are only schematic representations of embodiments that are given by way of non-limiting examples. For the purpose of clarity and a concise description, features are described herein as part of the same or separate embodiments, however, it will be appreciated that the scope of the disclosure may include embodiments having combinations of all or some of the features described.

The word ‘comprising’ does not exclude the presence of other features or steps. Furthermore, the words ‘a’ and ‘an’ shall not be construed as limited to ‘only one’, but instead are used to mean ‘at least one’, and do not exclude a plurality.

1. A method of filling a syringe with a liquid, comprising the steps of:
filling an intermediate volume of a buffer member with the liquid;
drawing the liquid from the intermediate volume of the buffer member into the main volume of the syringe such that the main volume is essentially free of air; and
disconnecting the buffer member from the syringe.

2. The method according to claim 1, wherein the intermediate volume of the buffer member is filled by drawing the liquid into the intermediate volume using a syringe connected to the buffer member.

3. The method according to claim 1, wherein the liquid is drawn into the intermediate volume through a filter needle.

4. The method according to claim 1, further comprising connecting a non-filter needle to the syringe after disconnecting the buffer member from the syringe.

5. The method according to claim 1, wherein the liquid is a liquid medicament.

6. The method according to claim 5, wherein the liquid medicament is filled into the intermediate volume from a glass ampoule or from a vial with a rubber stopper.

7. The method according to claim 1, wherein using the syringe, the liquid is first drawn into the main volume of the syringe, subsequently pushed into the intermediate volume of the buffer member, and then drawn from the intermediate volume of the buffer member back into the main volume of the syringe.

8. The method according to claim 1, wherein the volume of liquid drawn into the main volume of the syringe is less than 1 mL.

9. A method of filling a plurality of syringes with a liquid, comprising the steps of:
filling an intermediate volume of a buffer member with the liquid;
connecting one or more syringes to the buffer member; and
drawing liquid from the intermediate volume into main volumes of the one or more syringes.

10. The method according to claim 9, further comprising a step of connecting a filter needle to the buffer member, and wherein the liquid is drawn through the filter needle into the intermediate volume.

11. The method according to claim 9, wherein between 0.01 to 0.5 ml of liquid is drawn into the main volumes of each of the syringes.

12. (canceled)

13. A kit of parts for forming a syringe assembly, the kit of parts comprising:
a syringe with a main volume for receiving a liquid;
a buffer member, comprising a buffer body defining an intermediate volume for receiving a fluid into, a fluid inlet into the intermediate volume, and a vent opening for venting air from the intermediate volume;
wherein the buffer member is connectable to the syringe to form the syringe assembly, and, in a connected state, the main volume and the intermediate volume are in fluid communication.

14. The kit of parts according to claim 13, wherein:
the syringe comprises an elongated syringe body defining the main volume; and
a maximum cross-sectional area of the intermediate volume of the buffer body is larger than a maximum cross-sectional area of the main volume of the syringe in a direction perpendicular to an elongation direction of the syringe body.

15. The kit of parts according to claim 13, wherein the maximum cross-sectional area of the main volume is less than 25 mm² and the maximum cross-sectional area of the intermediate volume is less than 2·10³ mm², for example equal to or less than 1.2·10³ mm².

16. The kit of parts according to claim 13, wherein at least part of an inner surface of the buffer body facing the intermediate volume is a hydrophobic surface.

17. The kit of parts according to claim 13, wherein an inner surface of the buffer body facing the intermediate volume is essentially free of liquid lubricants.

18. (canceled)

19. The kit of parts according to claim 13, wherein when, in use, the fluid inlet and the vent opening are aligned in an approximately horizontal plane, the buffer member is arranged for holding a sub-volume of liquid in the intermediate volume below the fluid inlet and the vent opening, which sub-volume approximately corresponds to at least 0.2 times the size of the main volume of the syringe.

20. A buffer member for use in the method according to claim 1, comprising:
a buffer body defining an intermediate volume for receiving a fluid into;
a fluid inlet into the intermediate volume; and
a vent opening for venting air from the intermediate volume,
wherein the fluid inlet is arranged to be connected to a tip of a syringe.

21. The buffer member according to claim 20, wherein at least part on an inner surface of the buffer body facing the intermediate volume is a hydrophobic surface.

22. The buffer member according to claim 20, wherein the intermediate volume of the buffer body is sterile.
